(19) United States
(12) Patent Application Publication
(10) Pub. No.: US 2026/0258367 A1
(43) Pub. Date: Sep. 3, 2026

(54) INDUCTION/DIFFERENTIATION MEDIUM FOR PRODUCING OF MESENCHYMAL STEM CELLS AND APPLICATION THEREOF

(71) Applicant: HUNAN MEIBO BIOMEDICAL CO., LTD, Changsha (CN)

(72) Inventors: E XIAO, Changsha (CN); Qiushi LIU, Changsha (CN)

(73) Assignee: HUNAN MEIBO BIOMEDICAL CO., LTD, Changsha (CN)

(21) Appl. No.: 19/653,185

(22) Filed: Apr. 21, 2026

Related U.S. Application Data
(63) Continuation of application No. PCT/CN2025/119998, filed on Sep. 9, 2025.

(30) Foreign Application Priority Data
Dec. 26, 2024 (CN) 202411932891.4

(51) Int. Cl. C12N 5/0775

(52) U.S. Cl. CPC C12N 5/0668; C12N 2500/25; C12N 2500/32; C12N 2500/38; C12N 2501/105; C12N 2501/115; C12N 2501/135; C12N 2501/15; C12N 2501/727; C12N 2502/115; C12N 2506/45; C12N 2533/50; C12N 2533/52

(57) ABSTRACT

The present disclosure belongs to the field of stem cell biology, relates to lineage-specific differentiation of human pluripotent or embryonic stem cells, and specifically relates to a induction/differentiation medium for producing of mesenchymal stem cells and application thereof. The induction/differentiation medium is used for directly inducing differentiation of human induced pluripotent stem cells or embryonic stem cells into mesenchymal stem cells. The induction/differentiation medium includes a mesenchymal stem cell basal medium and additives. The additives include: 2 to 10 μM of a TGF-β inhibitor, 2 to 10 μM of a GSK3β inhibitor, and 5 to 10 μM of a ROCK inhibitor.

### CROSS-REFERENCE TO RELATED APPLICATIONS

This application is a Continuation of International Application No. PCT/CN2025/119998, filed on Sep. 9, 2025, which claims priority to Chinese Patent Application No. 202411932891.4, filed on Dec. 26, 2024, the entire contents of each of which are hereby incorporated by reference.

### TECHNICAL FIELD

The present disclosure generally relates to the field of stem cell biology, and in particular to lineage-specific differentiation of human induced pluripotent stem cells or embryonic stem cells, and specifically relates to a induction/differentiation medium for producing of mesenchymal stem cells and a preparation method thereof.

### BACKGROUND

Mesenchymal stem cells (MSCs) can be isolated from various human tissues, such as bone marrow, adipose tissue, umbilical cord blood, peripheral blood, neonatal tissue umbilical cord, placenta, or the like. However, a count of MSCs obtainable from adult tissues is limited, and an invasive procedure is required to isolate the MSCs, which also brings unexpected risks to a donor. Therefore, obtaining MSCs in sufficient quantity, with uniformity and stable quality, is key to realizing uses of the MSCs.

Human induced pluripotent stem cells include human embryonic stem cells (hESCs) and human induced pluripotent stem cells (iPSCs). The iPSCs can proliferate indefinitely in vitro while maintaining a potential to differentiate into almost all adult cell types. Directed differentiation of the iPSCs to obtain induced mesenchymal stem cells (iMSCs) provides a stable source of seed cells for MSC-based cell therapy. At present, numerous studies have reported acquisition of the MSCs using this strategy. Compared with tissue-derived MSCs, the iMSCs exhibit advantages such as higher homogeneity, more stable biological properties, and more predictable biological functions. However, MSCs are rare and exhibit heterogeneity among different tissues and organs, and the acquisition generally requires invasive procedures (except for those derived from umbilical cord), which limits potential of the MSCs for clinical translation. In addition, the MSCs have limited expansion capacity during culture and typically begin to undergo senescence after approximately 8 to 10 passages, making the MSCs difficult to generate a sufficient quantity of cells.

Therefore, a simple, reliable, and sufficient cell source is crucial for the translational application of the MSCs. The iPSCs are pluripotent stem cells obtained by inducing the adult cells through reprogramming technology, and possess characteristics similar to embryonic stem cells. iPSCs can be cultured and expanded in vitro without limitation, thereby continuously generating a large quantity of adult stem cells for use. Therefore, the iPSCs have gradually become an optimal cell source for obtaining the MSCs. At present, most established manners for differentiating the iPSCs into various cell types, including the MSCs, are based on spontaneous differentiation of embryoid bodies (EBs). Such manners usually require 30 to 40 days, with long processing time, low efficiency, and poor controllability. Another manner involves first inducing pluripotent stem cells to form trophoblast stem cells and subsequently differentiating the trophoblast stem cells into the MSCs. Such manners have problems such as incomplete detachment of upper-layer cells, long processing time, presence of mixed cell populations, insufficient differentiation, and low purity of the MSCs. Meanwhile, most existing disclosed manners for preparing the MSCs through directed induction of the iPSCs require adherent culture, enzymatic digestion, repeated passaging, and processes such as mouse-derived cell co-culture, coating with xenogeneic materials, flow cytometric sorting, or viral transfection. Such manners involve complicated and cumbersome operations, produce low yields with unstable quality, and are not suitable for large-scale production. Moreover, the introduction of exogenous xenogeneic substances during the culture process further limits practical applicability of the iPSCs.

Based on this, the present disclosure aims to provide a medium for induction and differentiation of the MSCs and a use thereof.

### SUMMARY

A first aspect of the present disclosure provides a induction/differentiation medium for producing of mesenchymal stem cells. The induction/differentiation medium is used for directly inducing differentiation of human induced pluripotent stem cells or embryonic stem cells into mesenchymal stem cells. The induction/differentiation medium includes a mesenchymal stem cell basal medium and additives. The additives include: 2 to 10 μM of transforming growth factor-β (TGF-β) inhibitor, 2 to 10 μM of glycogen synthase kinase-3β (GSK3β) inhibitor, and 5 to 10 μM of rho-associated coiled-coil containing protein kinase (ROCK) inhibitor.

A second aspect of the present disclosure further provides a method for directly inducing and differentiating human induced pluripotent stem cells or embryonic stem cells into mesenchymal stem cells. The method includes subjecting the human induced pluripotent stem cells or the embryonic stem cells to passaging culture in the induction/differentiation medium according to the first aspect of the present disclosure, thereby directly inducing and differentiating the human induced pluripotent stem cells or the embryonic stem cells into the mesenchymal stem cells.

A third aspect of the present disclosure further provides mesenchymal stem cells prepared by the method for directly inducing and differentiating human induced pluripotent stem cells or embryonic stem cells into mesenchymal stem cells provided according to the second aspect of the present disclosure.

In some embodiments of the present disclosure, the mesenchymal stem cell is a multipotent cell capable of differentiating into an adipocyte, an osteocyte, a chondrocyte, a myocyte, a neuron, and a cardiomyocyte.

Compared with the prior art, the present disclosure has the following beneficial effects:

(1) The present disclosure provides a medium for directly inducing and differentiating human induced pluripotent stem cells into mesenchymal stem cells and a method for directly inducing and differentiating human induced pluripotent stem cells into mesenchymal stem cells. By using the induction/differentiation medium for producing of mesenchymal stem cells provided by the present disclosure, human induced pluripotent stem cells can be directly induced to differentiate into mesenchymal stem cells. A cell differentiation pathway is clear, a differentiation efficiency is high, and a differentiation effect is stable. A culture system containing serum or feeder cells is not used. The obtained cell population has high purity and large quantity, thereby addressing the problems in the prior art, such as long induction and differentiation time, long passaging interval, and slow cell proliferation. The serum-free and xeno-free culture system solves safety problems. Moreover, even after repeated passage culture, characteristics of the mesenchymal stem cells can be maintained over a long period.

(2) The present disclosure significantly improves a differentiation efficiency of the mesenchymal stem cells, and even after long-term passage culture (for example, 12 passages, or even 15 passages or more), the obtained cells still exhibit excellent performance in stably maintaining characteristics of the mesenchymal stem cells. Through such passaging, mesenchymal stem cells derived from the human induced pluripotent stem cells can be prepared in large quantities. During the differentiation process, differentiation pathways are precisely controlled through combined use of small molecule compounds, achieving stable and efficient differentiation, and finally obtaining mature MSCs. The obtained MSCs express cell surface markers Cluster of Differentiation (CD) 90, CD73, CD105, and do not express CD14, CD34, CD45, CD19, and Human Leukocyte Antigen-DR isotype (HLA-DR). The MSCs grow in an adherent manner and possess osteogenic and adipogenic differentiation potential, without requiring additional flow cytometric sorting for cell selection.

(3) Compared with an induction manner disclosed in the prior art where pluripotent stem cells are first induced to trophoblast stem cells and the trophoblast stem cells are subsequently differentiated into mesenchymal stem cells, such approaches suffer from problems including incomplete detachment of upper-layer cells, long processing time, the presence of mixed cell populations, insufficient differentiation, and low purity of mesenchymal stem cells. By using the induction/differentiation medium provided in the present disclosure, the human induced pluripotent stem cells can be directly induced to differentiate into the mesenchymal stem cells.

The process described herein requires a shorter time and establishes an induction and differentiation system with clearly defined components. A serum-free culture system enables directed induction and differentiation to efficiently obtain mesenchymal stem cells with uniform purity and stable performance. Compared with the spontaneous differentiation of EBs disclosed in the prior art, which has problems such as low induction efficiency, possible retention of other pluripotent cells, long processing time, labor-intensive procedures, low efficiency, and poor controllability, the induction and differentiation medium provided herein enables direct differentiation of the human induced pluripotent stem cells into the mesenchymal stem cells, thereby simplifying the experimental procedure and providing good reproducibility. That is, compared with prior art manners involving spontaneous differentiation through EB formation or induction in which the pluripotent stem cells are first converted into trophoblast stem cells and then differentiated into the mesenchymal stem cells, the method provided in the present disclosure requires a shorter time and uses a defined-component serum-free induction and differentiation system to achieve efficient directed monolayer differentiation, thereby obtaining mesenchymal stem cells with uniform purity and stable performance.

### BRIEF DESCRIPTION OF THE DRAWINGS

To describe the technical solutions in the embodiments of the present disclosure more clearly, the following briefly introduces the accompanying drawings required for describing the embodiments. It should be understood that the following drawings illustrate only certain embodiments of the present disclosure and therefore should not be considered as limiting the scope of the present disclosure.

FIG. 1 is a schematic diagram illustrating pluripotency identification results of human induced pluripotent stem cells according to Example 1 of the present disclosure.

FIG. 2 is a schematic diagram illustrating a morphology of iPSC-derived iMSCs at different passages obtained according to Example 2 of the present disclosure.

FIG. 3A is a schematic diagram illustrating proportions of CD90+, CD73+, CD105+, and CD45− in iMSCs detected by flow cytometry according to some effect examples of the present disclosure.

FIG. 3B is a schematic diagram illustrating proportions of CD14−, CD34−, CD19−, and HLA-DR-phenotypes in iMSCs detected by flow cytometry according to some effect examples of the present disclosure.

FIG. 4 is a schematic diagram illustrating detection results of expression of MSC-related marker genes OCT4, SNAI2, COL6A2, and TWIST1 by qRT-PCR according to some effect examples of the present disclosure.

FIG. 5 is a schematic diagram illustrating detection results of expression of osteogenic-specific genes OCN, ALP, and RUNX2 in iMSCs according to some effect examples of the present disclosure.

FIG. 6 is a schematic diagram illustrating detection results of osteogenic differentiation capability of iMSCs according to some effect examples of the present disclosure.

FIG. 7 is a schematic diagram illustrating detection results of expression of adipogenic-specific genes PPARγ2 and LPL in iMSCs according to some effect examples of the present disclosure.

FIG. 8 is a schematic diagram illustrating detection results of adipogenic differentiation capability of iMSCs according to some effect examples of the present disclosure.

FIG. 9 is a schematic diagram illustrating cell culture states of No. 1 to No. 6 different induction and differentiation media in comparative examples of the present disclosure.

FIG. 10 is a schematic diagram illustrating cell states on day 6 of differentiation using iPSC-iMSC induction and differentiation media of Group A, Group B, and Group C according to comparative examples of the present disclosure.

### DETAILED DESCRIPTION

To facilitate understanding of the present disclosure, a more comprehensive and detailed description of the present disclosure is provided below in conjunction with the accompanying drawings and preferred embodiments. It should be noted that the described embodiments are only a part of the embodiments of the present disclosure, and not all of the embodiments. Based on the embodiments in the present disclosure, all other embodiments obtained by those of ordinary skill in the art without creative efforts shall fall within the scope of protection of the present disclosure.

A first aspect of the present disclosure provides a induction/differentiation medium for producing of mesenchymal stem cells. The induction/differentiation medium is used for directly inducing differentiation of human induced pluripotent stem cells (iPSCs) or embryonic stem cells into mesenchymal stem cells (MSCs, also referred to as iMSCs). The induction/differentiation medium includes: a mesenchymal stem cell basal medium and additives. The additives include the following components: 2 to 10 μM of transforming growth factor-β (TGF-β) inhibitor, 2 to 10 μM of glycogen synthase kinase-3β (GSK3β) inhibitor, and 5 to 10 μM of rho-associated coiled-coil containing protein kinase (ROCK) inhibitor.

The induction/differentiation medium for producing of mesenchymal stem cells refers to a functional medium containing specific additives. For example, the induction/differentiation medium for producing of mesenchymal stem cells is a medium supplemented with specific signaling pathway regulators and nutritional additives.

The mesenchymal stem cell basal medium refers to a basal medium used to provide a basic nutritional environment for induction and differentiation culture or expansion culture of the MSC. For example, the mesenchymal stem cell basal medium is used to provide basic nutrients such as amino acids, vitamins, inorganic salts, glucose, and a buffer system necessary for cell growth, and the mesenchymal stem cell basal medium does not specifically refer to a medium containing specific functional additives.

In some embodiments, the mesenchymal stem cell basal medium is selected from a serum-free basal medium for mesenchymal stem cells. The serum-free basal medium for mesenchymal stem cells includes at least one of high-glucose dulbecco's modified eagle medium (DMEM), alpha minimum essential medium (Alpha-MEM), or dulbecco's modified eagle medium/ham's f-12 nutrient mixture (DMEM/F12) medium.

The serum-free basal medium for mesenchymal stem cells refers to a basal medium that does not contain animal-derived serum components (e.g., fetal bovine serum (FBS)), and the serum-free basal medium for mesenchymal stem cells is used to provide a basic nutritional support environment for induction and differentiation culture or expansion culture of the MSC. For example, the serum-free basal medium for mesenchymal stem cells includes, but is not limited to, amino acids, vitamins, inorganic salts, glucose, the buffer system, and other conventional medium components, and the serum-free basal medium for mesenchymal stem cells does not contain animal serum.

The high-glucose DMEM refers to a high-glucose type Dulbecco's Modified Eagle Medium, which is one of commonly used basal medium types for mammalian cells. In some embodiments, the high-glucose DMEM may be a DMEM medium with a glucose concentration of about 4.5 g/L (about 25 mM), and the high-glucose DMEM is used to provide a high-energy supply environment for cells.

The Alpha-MEM refers to a medium formed by modifying a MEM medium. In some embodiments, the Alpha-MEM typically contains richer amino acids, vitamins, nucleotides, and other nutrients, and the Alpha-MEM is suitable for culture of various adherent cells, especially for culture of fibroblast-like cells and the MSCs.

The DMEM/F12 medium refers to a composite basal medium formed by mixing a Dulbecco's Modified Eagle Medium and a Ham's F12 Nutrient Mixture in a certain ratio. For example, the DMEM/F12 medium generally includes basic medium components such as amino acids, vitamins, inorganic salts, glucose, trace elements, and the buffer system, and does not contain the animal serum, and therefore can be used as a basal culture solution for a serum-free culture system.

In some embodiments of the present disclosure, by establishing a culture system free of the animal serum and xenogeneic animal components, batch-to-batch variations of the animal serum and risks of pathogen contamination are eliminated, and clinical safety of cell therapy products is significantly improved.

The additives refer to functional components additionally added to the mesenchymal stem cell basal medium. In some embodiments, the additives may be used to regulate cell signaling pathways, promote cell induction and differentiation, enhance cell proliferation capacity, or maintain stability of the cell phenotype.

The TGF-β inhibitor refers to a compound or a biologically active molecule that inhibits an activity of a transforming growth factor-β (TGF-β) signaling pathway. In some embodiments, the TGF-β inhibitor is able to block a TGF-β signal transduction process by inhibiting phosphorylation of a TGF-β type I receptor kinase (e.g., Activin Receptor-Like Kinase 5 (ALK5)) or related downstream signaling molecules. In some embodiments, the TGF-β inhibitor has a regulatory effect on differentiation of pluripotent stem cells.

In some embodiments, in the induction/differentiation medium for producing of mesenchymal stem cells, the dosage of the TGF-β inhibitor may be 2 μM, 3 μM, 4 μM, 5 μM, 6 μM, 7 μM, 8 μM, 9 μM, 10 μM, or a range formed by any two of the above values. The dosage is not limited to the listed values, and other unlisted values within the range are also applicable.

The GSK3β inhibitor refers to a compound or a biologically active molecule capable of inhibiting an activity of a glycogen synthase kinase-3 beta (GSK3β). In some embodiments, the GSK3β inhibitor promotes β-catenin stabilization and activates a Wnt/β-catenin signaling pathway by inhibiting a kinase activity of the GSK3β, thereby regulating stem cell proliferation, survival, and lineage differentiation processes.

In some embodiments, in the induction/differentiation medium for producing of mesenchymal stem cells, a dosage of the GSK3β inhibitor may be 2 μM, 3 μM, 4 μM, 5 μM, 6 μM, 7 μM, 8 μM, 9 μM, 10 μM, or a range formed by any two of the above values. The dosage is not limited to the listed values, and other unlisted values within the range are also applicable.

The ROCK inhibitor refers to a compound or a biologically active molecule that inhibits an activity of a rho-associated coiled-coil containing protein kinase (ROCK). In some embodiments, an addition of the ROCK inhibitor reduces an incidence of apoptosis after cell dissociation, improves a survival rate of single cells, and helps maintain adherent growth and stable expansion of cells.

In some embodiments, in the induction/differentiation medium for producing of mesenchymal stem cells, a dosage of the ROCK inhibitor may be 5 μM, 6 μM, 7 μM, 8 μM, 9 μM, 10 μM, or a range formed by any two of the above values. The dosage is not limited to the listed values, and other unlisted values within the range are also applicable.

In some embodiments, the TGF-β inhibitor includes at least one of SB431542, A-8301, or LY2157299. The GSK3β inhibitor includes at least one of CHIR99021, SB415286, or LY2090314. The ROCK inhibitor includes at least one of Y-27632 or Blebbistatin.

The SB431542 refers to a selective TGF-β signaling pathway inhibitor, which is a small molecule compound that inhibits a kinase activity of a TGF-β type I receptor kinase and related receptors. In some embodiments, the SB431542 may inhibit transduction of TGF-3/Activin/Nodal signaling pathways by blocking a phosphorylation process of Mothers against decapentaplegic homolog 2/Mothers against decapentaplegic homolog 3 (Smad2/Smad3). In some embodiments, the SB431542 may be used to inhibit an activity of the TGF-β signaling pathway, thereby promoting differentiation of the iPSCs or the embryonic stem cells into a mesenchymal lineage.

The A-8301 refers to a chemical small molecule inhibitor that selectively inhibits an activity of the ALK5. In some embodiments, the A-8301 may inhibit a phosphorylation process of the Smad2/Smad3 in the TGF-β signaling pathway, thereby blocking the TGF-β signal transduction process. In some embodiments, the A-8301 may be added exogenously to the mesenchymal stem cell basal medium for constructing the induction/differentiation medium for producing of mesenchymal stem cells.

The LY2157299 refers to an ALK5 inhibitor. In some embodiments, the LY2157299 specifically inhibits the activity of the ALK5, thereby blocking downstream signal transduction of the TGF-β signaling pathway (e.g., phosphorylation of the Smad2/Smad3), and regulating cell proliferation, differentiation, and apoptosis. In some embodiments, the LY2157299 may be added as an exogenous TGF-β inhibitor to the mesenchymal stem cell basal medium for constructing the induction/differentiation medium for producing of mesenchymal stem cells, to promote direct differentiation of the iPSCs or the embryonic stem cells into the MSCs.

The CHIR99021 refers to a small molecule compound capable of selectively inhibiting a kinase activity of the GSK3β and activating the Wnt/β-catenin signaling pathway. In some embodiments, the CHIR99021 may promote proliferation of pluripotent stem cells, improve a survival rate, and regulate a lineage differentiation direction of the pluripotent stem cells by stabilizing β-catenin. In some embodiments, the CHIR99021 may be added exogenously as one of the GSK3β inhibitors to the mesenchymal stem cell basal medium for constructing the induction/differentiation medium for producing of mesenchymal stem cells, thereby promoting direct differentiation of the iPSCs or the embryonic stem cells into the MSCs.

The SB415286 refers to a compound capable of selectively inhibiting the kinase activity of the GSK3β. In some embodiments, the SB415286 may bind to an ATP-binding site of the GSK3β to inhibit an ability thereof to phosphorylate substrates, thereby stabilizing the β-catenin and activating the Wnt/β-catenin signaling pathway. In some embodiments, the SB415286 may promote stem cell proliferation, survival, and lineage-specific differentiation through the Wnt/β-catenin signaling pathway.

The LY2090314 refers to a small chemical molecule capable of specifically inhibiting the kinase activity of the GSK3β. In some embodiments, the LY2090314 may serve as a GSK3β inhibitor in the induction/differentiation medium for producing of mesenchymal stem cells, synergizing with the TGF-β inhibitor and the ROCK inhibitor to promote directed differentiation of the iPSCs or the embryonic stem cells toward the mesenchymal lineage. The LY2090314 includes commercially available or self-prepared compounds having a GSK3β inhibitory effect, and a source and a specific production manner of the LY2090314 are not limited.

The Y-27632 refers to a small molecule compound capable of selectively inhibiting an activity of the ROCK. In some embodiments, the Y-27632 may regulate cytoskeleton reorganization and reduce an apoptosis rate after the cell dissociation, thereby increasing the survival rate of the single cells. In some embodiments, the Y-27632 may be used to stabilize adherent growth and proliferation of the MSCs. The Y-27632 includes commercially available or self-synthesized small molecule compounds having a ROCK inhibitory effect, and a source and a specific manufacturer of the Y-27632 are not limited.

The Blebbistatin refers to a small molecule compound capable of selectively inhibiting an adenosine triphosphatase (ATPase) activity of non-muscle myosin II. In some embodiments, the Blebbistatin may indirectly inhibit downstream functions associated with a ROCK signaling pathway. In some embodiments, during stem cell culture and passage, the Blebbistatin reduces apoptosis of dissociated cells, improves the survival rate of the single cells, and helps maintain adherent growth and stable proliferation of cells.

In some embodiments of the present disclosure, by selectively adding the TGF-β inhibitor (e.g., the SB431542, the A-8301, and the LY2157299), the GSK3β inhibitor (e.g., the CHIR99021, the SB415286, and the LY2090314), and the ROCK inhibitor (e.g., the Y-27632 and the Blebbistatin) to the induction/differentiation medium for producing of mesenchymal stem cells, activities of TGF-β, Wnt/β-catenin, and Rho/ROCK signaling pathways are regulated, thereby effectively promoting directly directed differentiation of the iPSCs or the embryonic stem cells into the MSCs, while helping to improve a survival rate of the cells and proliferation capacity, and obtaining P3~P5 (Passage 3~Passage 5) passage MSCs with a stable morphology and phenotype.

In some embodiments, the additives further include: human platelet lysate, ascorbic acid, GlutaMax additive, non-essential amino acids (NEAA), insulin-transferrin-selenium-ethanolamine (ITS-X) supplement, Insulin-like Growth Factor (IGF), basic fibroblast growth factor (bFGF), and platelet-derived growth factor-BB (PDGF-BB).

In some embodiments, the human platelet lysate is at a volume concentration of 1% to 10%, the ascorbic acid is at a concentration of 30 to 70 mM, the GlutaMax additive is at a volume concentration of 0.5% to 2%, the NEAA is at a volume concentration of 0.5% to 2%, the ITS-X supplement is at a volume concentration of 0.5% to 2%, the IGF is at a concentration of 1 to 10 ng/mL, the bFGF is at a concentration of 1 to 10 ng/mL, and the PDGF-BB is at a concentration of 1 to 10 ng/mL.

The human platelet lysate (hPL) refers to a complex obtained after physical or chemical disruption of human platelets and enriched in platelet growth factors, cytokines, and other soluble bioactive molecules. In some embodiments, the hPL may replace animal serum to provide growth factors for the MSCs, promote proliferation, maintain the stability of the cell phenotype, and support stem cell expansion and induced differentiation in the serum-free culture system. The hPL may be obtained from platelets of healthy human donors, prepared by manners such as freeze-thaw cycles, ultrasonic treatment, or chemical lysis, and may undergo treatments such as virus inactivation or fibrin removal to improve safety and reproducibility.

In some embodiments, in the induction/differentiation medium for producing of mesenchymal stem cells, a volume concentration of the hPL may be 1%, 2%, 3%, 4%, 5%, 6%, 7%, 8%, 9%, 10%, or a range formed by any two of the above values, and is not limited to the listed values, and other unlisted values within the range are also applicable.

In some embodiments, in the induction/differentiation medium for producing of mesenchymal stem cells, a dosage of the ascorbic acid may be 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, or a range formed by any two of the above values, and is not limited to the listed values, and other unlisted values within the range are also applicable.

The GlutaMax additive refers to a cell culture supplement providing a stabilized source of L-glutamine, used to support cell growth, proliferation, and metabolism. For example, the GlutaMax additive may be a stable derivative of L-glutamine (e.g., L-alanyl-L-glutamine dipeptide), which gradually releases active glutamine in a culture medium, reduces ammonia accumulation during culture, and improves the cell growth environment. In some embodiments, the GlutaMax additive is obtained from commercially available cell culture-grade L-alanyl-L-glutamine dipeptide preparations.

In some embodiments, in the induction/differentiation medium for producing of mesenchymal stem cells, a volume concentration of the GlutaMax additive may be 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2%, or a range formed by any two of the above values, and is not limited to the listed values, and other unlisted values within the range are also applicable.

The NEAA refer to amino acids that mammalian cells can synthesize in vivo, and are used as nutritional supplement components in cell culture media to maintain basic amino acid levels required for cell growth, proliferation, and differentiation. In some embodiments, the NEAA may assist stem cells in maintaining a healthy proliferative state.

In some embodiments, in the induction/differentiation medium for producing of mesenchymal stem cells, a volume concentration of the NEAA may be 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2%, or a range formed by any two of the above values, and is not limited to the listed values, and other unlisted values within the range are also applicable.

The ITS-X supplement refers to a chemical nutritional additive commonly used in mammalian cell culture and mainly used to provide insulin, transferrin, selenium, and other necessary cofactors to support cell proliferation and maintain the stability of the cell phenotype.

The ITS-X supplement includes commercial or self-prepared equivalent formulations providing nutrients such as insulin, transferrin, and selenium, and a specific source and a composition of the ITS-X supplement are not limited.

In some embodiments, in the induction/differentiation medium for producing of mesenchymal stem cells, a volume concentration of the ITS-X supplement may be 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2%, or a range formed by any two of the above values, and is not limited to the listed values, and other unlisted values within the range are also applicable.

The IGF refers to a class of polypeptide growth factors capable of promoting cell proliferation, differentiation, and survival. The IGF may be Insulin-like Growth Factor 1 (IGF-1), IGF-2, or the like. The IGF includes insulin-like growth factors having functions of promoting cell proliferation and differentiation, and a specific subtype and source of the IGF are not limited.

In some embodiments, in the induction/differentiation medium for producing of mesenchymal stem cells, a dosage of the IGF may be 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, or a range formed by any two of the above values, and is not limited to the listed values, and other unlisted values within the range are also applicable.

The bFGF refers to a multifunctional growth factor widely present in mammalian tissues, belonging to a fibroblast growth factor (FGF) family. In some embodiments, the bFGF may bind to FGF receptors (FGFRs) and activate downstream signaling pathways, regulating stem cell proliferation, survival, and differentiation. The bFGF may be obtained from recombinant human bFGF products meeting cell culture-grade standards, and a source and manufacturer of the bFGF are not limited.

In some embodiments, in the induction/differentiation medium for producing of mesenchymal stem cells, a dosage of the bFGF may be 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, or a range formed by any two of the above values, and is not limited to the listed values, and other unlisted values within the range are also applicable.

The PDGF-BB refers to one of dimeric forms of platelet-derived growth factor (PDGF), which is a homodimer composed of two B chains of the PDGF. In some embodiments, the PDGF-BB may be added exogenously to the mesenchymal stem cell basal medium or an induction and differentiation medium for constructing the induction/differentiation medium for producing of mesenchymal stem cells or an expansion medium for mesenchymal stem cells. In some embodiments, the PDGF-BB may promote cell proliferation, migration, and differentiation by binding to PDGF receptors and activating downstream signaling pathways of the PDGF receptors, including a PI3K/Akt signaling pathway, an MAPK signaling pathway, etc. In mesenchymal stem cell culture, the PDGF-BB may be used to enhance cell proliferation capacity, maintain cell viability, and support stability of a lineage differentiation direction. PDGF-BB includes PDGF-BB proteins from any source or functional equivalents thereof, and a specific source and purification manner of the PDGF-BB are not limited.

In some embodiments, in the induction/differentiation medium for producing of mesenchymal stem cells, a dosage of the PDGF-BB may be 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, or a range formed by any two of the above values, and is not limited to the listed values, and other unlisted values within the range are also applicable.

In some embodiments of the present disclosure, by adding a combination of effective additives verified through experiments to the induction/differentiation medium for producing of mesenchymal stem cells, the induction/differentiation medium not only provides comprehensive nutritional and growth factor support for the cells, but also promotes efficient differentiation of the iPSCs or the embryonic stem cells into the MSCs, increases cell proliferation speed, and maintains phenotypic stability and expression of pluripotency markers of the MSCs.

The direct induction of differentiation refers to a culture manner in which the iPSCs or the hESCs are directed to differentiate into a target cell type under specific culture conditions without undergoing embryoid body (EB) formation, feeder cell co-culture, or other intermediate cell lineage stages.

In some embodiments of the present disclosure, by simultaneously adding inhibitors of multiple signaling pathways to the mesenchymal stem cell basal medium, the medium can directly induce a differentiation of the iPSCs or the embryonic stem cells into the MSCs, thereby avoiding steps of the EB formation or the feeder cell co-culture, shortening a differentiation cycle, and significantly reducing a culture time and production costs. In some embodiments, by adopting a xeno-free, serum-free culture system and combining synergistic effects of a plurality of small molecule inhibitors, an efficient, synchronous, and uniform directed differentiation is achieved. The obtained induced mesenchymal stem cells (iMSCs) have a high purity and do not require flow purification by flow cytometric sorting, thereby avoiding cell damage and process complexity and significantly improving product uniformity and process reproducibility.

A second aspect of the present disclosure provides a method for direct induction and differentiation of human induced pluripotent stem cells (iPSCs) or embryonic stem cells into mesenchymal stem cells (MSCs). The method includes: performing passage culture on the iPSCs or the embryonic stem cells using the induction/differentiation medium for producing of mesenchymal stem cells according to the first aspect of the present disclosure, thereby directly inducing and differentiating the iPSCs or the embryonic stem cells into the MSCs.

In some embodiments, the method includes: step a. culturing a cell culture of the iPSCs or the embryonic stem cells; and step b. replacing a medium in the cell culture with the medium for induction and differentiation of MSCs to perform passaging culture, thereby obtaining P3~P5 passage MSCs or a cell culture including the P3~P5 passage MSCs.

The P3~P5 passage refers to a cell population obtained after cells have undergone 3 to 5 successive passages during in vitro culture, starting from an initial passage. For example, when a cell population that has been cultured in a culture medium to a predetermined confluence for the first time is designated as P0 passage, each subsequent completed passage culture is designated as P1 passage, P2 passage, P3 passage, etc.

The passage culture refers to a process in which, when cells grow to a certain confluence, the cells are separated by digestion or mechanical pipetting, and are transferred to a new culture container at a certain ratio for continued culture.

The cell culture refers to a cell population obtained under in vitro culture conditions and a culture system where the cell population is located.

In some embodiments of the present disclosure, by replacing the medium in the cell culture with the induction/differentiation medium for producing of mesenchymal stem cells, and performing the passage culture on the cell culture of the iPSCs or the embryonic stem cells, pluripotent stem cells can be directly induced to differentiate into the MSCs. This manner simplifies a traditional multi-step differentiation process, shortens a differentiation cycle, and significantly reduces culture time and production cost.

In some embodiments, in step a, the culturing the cell culture includes: culturing the iPSCs or the embryonic stem cells in a xeno-free medium in a feeder-free culture manner.

The step of culturing the cell culture further includes: first culturing the iPSCs or the embryonic stem cells normally in an induced pluripotent stem cell (iPSC) maintenance medium; when confluence of the cell culture reaches 80% to 90%, digesting the iPSCs with Tryple into a single-cell suspension; resuspending the single-cell suspension in an iPSC maintenance medium; adding the ROCK inhibitor to the iPSC maintenance medium; after maintaining the ROCK inhibitor for 24 h, and replacing with a complete iPSC maintenance medium (also referred to as an iPSC complete medium) to obtain the cell culture at 30% to 50% confluence.

In some embodiments, the iPSC maintenance medium includes at least one of Chemically Defined Essential 8 Medium (E8), StemFit Basic04, or mTeSR Plus.

In some embodiments, the ROCK inhibitor includes at least one of Y-27632 or Blebbistatin.

In some embodiments, a matrix gel used in the culturing the cell culture includes at least one of Laminin-521, Vitronectin, or Matrigel.

In some embodiments, culture conditions for the step of culturing the cell culture include: culturing in an incubator at 35-40° C., 4-6% CO₂, and 92-98% humidity.

The xeno-free medium refers to a medium in which the medium and related additives do not contain components derived from non-human animals, such as FBS, horse serum, fetal bovine serum-derived proteins, and other biological products from non-human mammals.

The culturing normally refers to a process of maintaining or expanding cells under conventional cell culture conditions using a basal medium suitable for the cell type and conventional culture parameters. For example, the culturing normally does not include a special induction culture process performed by artificially applying a specific induction factor (e.g., a directed differentiation induction factor, a strong stimulation factor, a chemical inducer, etc.).

The confluence refers to a percentage of a surface area of a culture container covered by adherently growing cells relative to a cultivable surface area, and is used to determine whether the cells have reached an appropriate timing for passage or an induction treatment. For example, when cells grow in a monolayer on a bottom of a culture container, a coverage area gradually increases as the cells proliferate. When the cells essentially cover the entire cultivable surface, the confluence is considered to be 100%.

The Tryple refers to a recombinant enzyme-based cell dissociation reagent used to digest adherent cells, allowing the adherent cells to detach from a surface of a culture container and disperse into single cells or cell aggregates. In some embodiments, the Tryple may be used for passaging, digestion, or cell collection of the MSCs.

The single-cell suspension refers to a cell suspension system in which cells are uniformly dispersed as single cells in a liquid medium or buffer after digestion, pipetting, or filtration treatment, and the single-cell suspension substantially contains no cell clusters visible to naked eyes or identifiable under a microscope.

The iPSC maintenance medium refers to a medium used to maintain the iPSCs in an undifferentiated state with self-renewal capacity and pluripotency characteristics under in vitro culture conditions.

The complete iPSC maintenance medium refers to a conventional iPSC maintenance medium without the ROCK inhibitor, and the complete iPSC maintenance medium may be used to maintain normal growth and a pluripotency state of the iPSCs or the embryonic stem cells.

The E8 refers to a serum-free culture medium composed of eight core components. In some embodiments, the E8 typically includes a DMEM/1F12 basal medium, L-ascorbic acid, selenium, transferrin, insulin, recombinant human bFGF, recombinant human TGF-β1, and other essential additives. In some embodiments, the E8 may be used to maintain an undifferentiated state of the iPSCs or the embryonic stem cells.

The StemFit Basic04 refers to a basal medium product used to maintain an undifferentiated state of the iPSCs. The StemFit Basic04 is typically a serum-free, chemically defined, or animal component-free medium used to support the iPSCs in maintaining the self-renewal capacity and the pluripotency characteristics during in vitro culture.

The mTeSR Plus refers to a commercial, serum-free, immune-compatible iPSC maintenance medium used to maintain an undifferentiated state and pluripotency of the iPSCs in vitro. In some embodiments, the mTeSR Plus may be used for iPSC maintenance culture, single-cell passage, or the like.

The matrix gel refers to an extracellular matrix coating material used for adherent cell culture, the primary function of which is to provide a supportive environment required for cell attachment, expansion, and maintenance of pluripotency or a specific differentiated state. For example, the matrix gel may be a recombinant human Laminin protein, a recombinant human Vitronectin protein, a composite matrix protein extracted from mouse EHS sarcoma, etc. In some embodiments, the matrix gel used in the culture process includes at least one of Laminin alpha-5 beta-2 gamma-1(Laminin-521), Vitronectin, or Matrigel Basement Membrane Matrix (Matrigel). In some embodiments, the matrix gel may be used in iPSC maintenance culture, MSC induction and differentiation, and cell expansion processes.

Matrigel refers to a natural composite matrix gel derived from a mouse tumor (Engelbreth-Holm-Swarm, EHS tumor) matrix, used to provide an adherent support and a three-dimensional matrix microenvironment during cell culture. Matrigel is a murine-derived material and does not belong to a xeno-free culture system.

The culturing conditions refer to physical and chemical environmental parameters adopted when culturing the cell culture. The culturing conditions include, but are not limited to, temperature, CO₂ concentration, humidity, a medium composition, and a culture container. In some embodiments, a temperature for the step of culturing the cell culture needs to be controlled within a certain range. In some embodiments, the temperature for the step of culturing the cell culture may be 35° C., 36° C., 37° C., 38° C., 39° C., 40° C., or any value within a range formed by any two of the above values. In some embodiments, a CO₂ concentration for the step of culturing the cell culture may be 4%, 5%, 6%, or any value within a range formed by any two of the above values. In some embodiments, a humidity for the step of culturing the cell culture needs to be controlled within a certain range. In some embodiments, the humidity for the step of culturing the cell culture may be 92%, 93%, 94%, 95%, 96%, 97%, 98%, or any value within a range formed by any two of the above values.

In some embodiments of the present disclosure, by culturing the iPSCs or the embryonic stem cells using the xeno-free medium, risks of animal-derived pathogens and immunogenicity can be avoided. By using the Tryple to digest the cells into the complete single-cell suspension when the cells reach 80% to 90% confluence, uniformity and viability of the digested cells can be improved. By adding the ROCK inhibitor to the iPSC maintenance medium and maintaining the ROCK inhibitor for 24 hours, an apoptosis rate of the cells during a single-cell suspension process can be significantly reduced, and adherent survival capability of the single cells can be improved. By performing adherent culture using the matrix gels such as Laminin-521, Vitronectin, or Matrigel, adherent growth of the cells can be promoted, and the undifferentiated state of the cells can be maintained. Meanwhile, by culturing the cells in the incubator at 35-40° C., 4-6% CO₂, and 92-98% humidity, stable proliferation of the cells in a suitable growth environment can be maintained.

In some embodiments, in step b, replacing the medium in the cell culture with the induction/differentiation medium for producing of mesenchymal stem cells to perform passaging culture includes: replacing the medium in the cell culture with the induction/differentiation medium for producing of mesenchymal stem cells; when confluence reaches 80%-90%, designating the cell culture as P0 passage MSCs; performing a digestion treatment on the P0 passage MSCs; resuspending the P0 passage MSCs after the digestion treatment using the induction/differentiation medium for producing of mesenchymal stem cells; continuing culture to obtain P1 passage MSCs; and repeating the above passage culturing steps to obtain P3~P5 passage MSCs.

In some embodiments, the digestion treatment includes: aspirating supernatant; adding Ca²⁺ and Mg²⁺-free dulbecco's phosphate buffered saline (DPBS) to wash the P0 passage MSCs; performing the digestion treatment on the P0 passage MSCs after washing; centrifuging to remove supernatant; and obtaining the P0 passage mesenchymal stem cells after the digestion treatment.

The digestion treatment refers to an operation process of detaching adherently growing cells from a surface of a culture container by enzymatic or chemical manners and dispersing a cell population into single cells or small cell aggregates. In some embodiments, the digestion treatment may be used to process the MSCs into the single cells or a small count of cell aggregates, to facilitate continuous passage in the induction/differentiation medium for producing of mesenchymal stem cells.

Resuspension refers to redispersing a cell pellet obtained after digestion or centrifugation in a liquid medium or buffer so that the cells are evenly distributed in a solution for subsequent experimental operations such as culture, counting, and seeding. In some embodiments, a manner for the resuspension may be gently pipetting the cell pellet or the cell aggregates with a pipette.

DPBS (Dulbecco's Phosphate-Buffered Saline) refers to a buffer system commonly used in the cell culture and experimental operations, and is used to maintain pH and osmotic pressure of extracellular fluid.

The Ca²⁺ and Mg²⁺-free DPBS refers to DPBS that does not contain calcium ions (Ca²⁺) and magnesium ions (Mg²⁺). In some embodiments, the Ca²⁺ and Mg²⁺-free DPBS may be used to wash cells before a cell digestion or passage operation to avoid promotion of cell adhesion or enhancement of tight intercellular junctions by Ca²⁺ and Mg²⁺.

In some embodiments of the present disclosure, by replacing the medium in the cell culture of the iPSCs or the embryonic stem cells with the induction/differentiation medium for producing of mesenchymal stem cells, and performing continuous passage culture on digested cells to the P3~P5 passage, and by controlling inhibition of the TGF-β signaling pathway, the GSK3β signaling pathway, and the ROCK signaling pathways, and supplementing with nutritional factors such as the human platelet lysate and the ascorbic acid, the iPSCs or the embryonic stem cells can be directly and efficiently induced to differentiating into the MSCs with the stable morphology, uniform phenotype, and a high proliferative activity.

In some embodiments, the method further includes: placing the P3~P5 passage MSCs or the cell culture including the P3~P5 passage MSCs in an expansion medium for mesenchymal stem cells to perform expansion passage culture, thereby obtaining the MSCs.

In some embodiments, the expansion medium for mesenchymal stem cells includes: the mesenchymal stem cell basal medium and the additives; the additives include: the human platelet lysate, the ascorbic acid, the GlutaMax additive, the NEAA, the ITS-X supplement, the IGF, the bFGF, and the PDGF-BB.

In some embodiments, the human platelet lysate is at a volume concentration of 1% to 10%, the ascorbic acid is at a concentration of 30 to 70 mM, the GlutaMax additive is at a volume concentration of 0.5% to 2%, the NEAA is at a volume concentration of 0.5% to 2%, the ITS-X supplement is at a volume concentration of 0.5% to 2%, the IGF is at a concentration of 1 to 10 ng/mL, the bFGF is at a concentration of 1 to 10 ng/mL, and the PDGF-BB is at a concentration of 1 to 10 ng/mL.

In some embodiments, the mesenchymal stem cell basal medium is a serum-free basal medium for mesenchymal stem cells. The serum-free basal medium for mesenchymal stem cells includes at least one of high-glucose DMEM, Alpha-MEM, or DMEM/F12 medium.

The expansion medium for mesenchymal stem cells refers to a medium that is based on the mesenchymal stem cell basal medium and supplemented with specific nutritional supplements and growth factors to promote stable proliferation and continuous passage of the MSCs in vitro.

In some embodiments of the present disclosure, by using an expansion medium containing specific additives at suitable concentrations (e.g., the human platelet lysate, the ascorbic acid, the GlutaMax, the NEAA, the ITS-X, the IGF, the bFGF, the PDGF-BB, etc.) for expansion passage culture of the P3~P5 passage MSCs or the cell culture including the P3~P5 passage MSCs, proliferative capacity and multipotent characteristics of the MSCs can be efficiently maintained, while stability of a cell morphology, surface markers, and differentiation potential can be preserved, thereby obtaining a sufficient quantity of MSCs with stable performance and providing a reliable cell source for subsequent scientific research or clinical uses.

In some embodiments of the present disclosure, by using the induction/differentiation medium for producing of mesenchymal stem cells provided in the present disclosure for the passage culture of the iPSCs or the embryonic stem cells, direct induction of cell differentiation toward the mesenchymal lineage can be achieved, the traditional multi-step differentiation process can be simplified, a differentiation efficiency and a cell yield can be improved, and MSCs with stable phenotypic characteristics and multipotent differentiation potential can be obtained, thereby facilitating promotion and use of the MSCs in scientific research and clinical uses.

A third aspect of the present disclosure further provides mesenchymal stem cells prepared by the method for direct induction and differentiation of iPSCs or embryonic stem cells into MSCs as provided in a second aspect of the present disclosure. The MSCs are multipotent cells capable of differentiating into an adipocyte, an osteocyte, a chondrocyte, a myocyte, a neuron, and a cardiomyocyte.

In some embodiments, the MSCs are capable of expressing cell surface markers Cluster of Differentiation (CD) 29, CD73, CD90, CD105, CD166, and CD44.

In some embodiments, the MSCs do not express cell surface markers HLA-DR, CD34, CD45, CD19, and CD14.

In some embodiments, the MSCs are P5 or higher passage MSCs. In the MSCs, a proportion of cells expressing CD29, CD44, CD73, CD90, CD105, and CD166 is not less than 95%. In the MSCs, a proportion of cells expressing HLA-DR, CD34, CD45, CD19, and CD14 is not more than 2%.

In some embodiments of the present disclosure, by preparing the MSCs using the method for direct induction and differentiation of iPSCs or embryonic stem cells into MSCs provided in the present disclosure, a cell population maintaining high purity, multipotency, and phenotypic stability can be obtained, thereby providing reliable cell resources for tissue engineering, regenerative medicine, and clinical cell therapy.

The technical solutions of the present disclosure are further illustrated below by specific examples. The specific examples do not represent a limitation on the scope of protection of the present disclosure. Some non-essential modifications and adjustments made by others based on the concept of the present disclosure still fall within the scope of protection of the present disclosure.

Information on cells and reagents used in the examples of the present disclosure is as follows:
Human induced pluripotent stem cells: Catalog number: hCiPSC-00409, Manufacturer: Beijing Beigi Biomedicine Co., Ltd.;
Laminin-521 stock solution: Catalog number: 200-0117, Manufacturer: STEMCELL Technologies Inc.;
DPBS (with calcium and magnesium): Catalog number: 14080055, Manufacturer: Gibco, Thermo Fisher Scientific Inc.;
DPBS (without calcium and magnesium): Catalog number: C14190500BT, Manufacturer: Gibco, Thermo Fisher Scientific Inc.;
Vitronectin stock solution: Catalog number: RP01002, Manufacturer: Sino Biological Inc.;
DMEM/F12: Catalog number: 11330-032, Manufacturer: Gibco, Thermo Fisher Scientific Inc.;
mTeSR Plus medium: Catalog number: 100-0276, Manufacturer: STEMCELL Technologies Inc.;
Tryple™ Express: Catalog number: 12604021, Manufacturer: Gibco, Thermo Fisher Scientific Inc.;
Y27632: Catalog number: HY-10071, Manufacturer: MedChemExpress LLC;
Versene digestion enzyme: Catalog number: 15040066, Manufacturer: Gibco, Thermo Fisher Scientific Inc.;
Human platelet lysate: Catalog number: PL-NH-100, Manufacturer: Sexton Biotechnologies, Inc.;
Ascorbic acid: Catalog number: A8960, Manufacturer: Merck KGaA;
GlutaMax additive: Catalog number: A1286001, Manufacturer: Gibco, Thermo Fisher Scientific Inc.;
NEAA: Catalog number: 11140050, Manufacturer: Gibco, Thermo Fisher Scientific Inc.;
ITS-X supplement: Catalog number: 51500056, Manufacturer: Gibco, Thermo Fisher Scientific Inc.;
IGF: Catalog number: GMP-C023, Manufacturer: ACROBiosystems Co., Ltd.;
bFGF: Catalog number: GMP-C046, Manufacturer: ACROBiosystems Co., Ltd.;
PDGF-BB: Catalog number: GMP-C199, Manufacturer: ACROBiosystems Co., Ltd.

### Example 1 Culture and Passage of Initial Cells

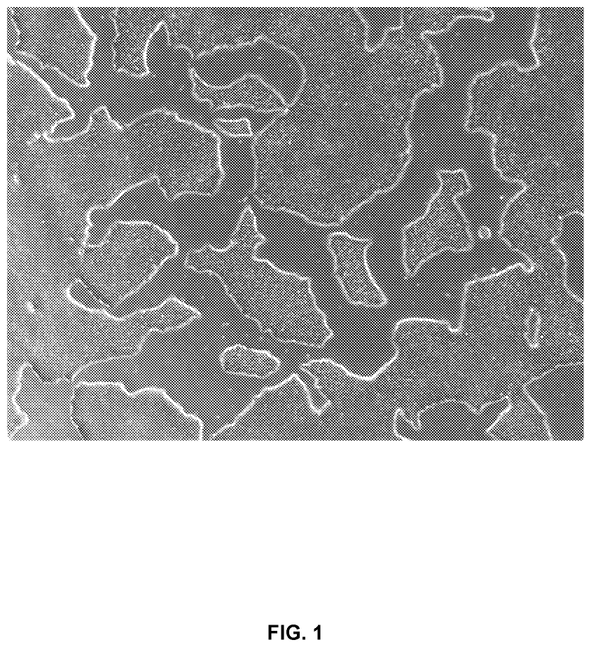

This example used iPSCs as initial cells. As specifically shown in FIG. 1, according to results shown in FIG. 1, the iPSCs exhibited typical colony growth characteristics, with clear colony edges, tight cell contacts within colonies, a high nuclear-to-cytoplasmic ratio, a uniform morphology, and no differentiated cells observed. Culture and passage operations for the iPSCs were as follows:

Coating a culture plate with Laminin-521: A Laminin-521 stock solution was taken, and the Laminin-521 was diluted to a final concentration of 5 g/mL using DPBS (with calcium and magnesium). The diluted Laminin-521 was added to the culture plate, and the culture plate was placed in a refrigerator at 2-8° C. overnight or at 37° C. for at least 2 hours for coating. After the coating was completed, the culture plate was taken out of the refrigerator and placed at a room temperature for 30 minutes before use.

Coating a culture plate with Vitronectin: A Vitronectin stock solution was taken, and the Vitronectin stock solution was diluted to a final concentration of 10 g/mL using DMEM/F12. The diluted Vitronectin was added to the culture plate, and the culture plate was placed in a refrigerator at 2-8° C. overnight or at a room temperature for at least 1 hour for coating. After the coating was completed, the culture plate was taken out of the refrigerator and placed at a room temperature for 30 minutes before use.

Cell thawing: A thawed iPSC suspension was transferred to a new 15 mL centrifuge tube, and an iPSC complete medium equilibrated to a room temperature was gently added dropwise to the iPSC suspension while gently shaking the centrifuge tube. The centrifuge tube containing the iPSC suspension was transferred to a low-speed refrigerated centrifuge and centrifuged at 300 g for 3 minutes at a room temperature. After centrifugation, supernatant was discarded, and an iPSC complete medium (mTeSR Plus) containing 10 μM Y27632 was added to resuspend the cells. The cells were gently pipetted and seeded at a volume ratio of 1:15 into a pre-warmed and prepared 12-well culture plate. The cells were cultured in an incubator at 37° C., 5% CO₂ concentration, and 95% humidity. The medium was changed every 20 to 24 hours. Cell passage was started when confluence of the cells reached more than 80%.

Cell passaging: When the confluence of the cells reached more than 80%, the culture plate was taken out of the incubator. The cells were washed once with DPBS (without calcium and magnesium). Then, a Versene digestion enzyme was added to the culture plate to digest the cells for 3 to 5 minutes. A state of colony dissociation was observed under a microscope. After the cells were completely dissociated, the iPSC complete medium (mTeSR Plus) was added and the cell suspension was gently pipetted to disperse the cells. Another blank culture plate pre-warmed to 37° C. was taken out of the incubator. The cell suspension was added at a volume ratio of 1:20. The culture plate was placed in the incubator at 37° C. and 5% CO₂ concentration for overnight culture. The medium was changed every 20 to 24 hours. A next round of the cell passaging was started when the confluence of the cells reached more than 80%. According to the above manner, the cells were passaged to P20 (Passage 20) to obtain P20 passage cells.

### Example 2

This example provides a preparation manner for direct induction and differentiation of iPSCs into MSCs, including:

Step a, human induced pluripotent stem cell culture: the iPSCs passaged to the P20 passage prepared in Example 1 were normally cultured in an iPSC maintenance medium. The iPSC maintenance medium used was mTeSR Plus. When the iPSCs were cultured to confluence of 80% to 90%, the iPSCs were digested into a complete single-cell suspension using Tryple. The cells were resuspended in the iPSC maintenance medium. A ROCK inhibitor was added to the iPSC maintenance medium. After the ROCK inhibitor was maintained for 24 hours, the medium was replaced with a complete iPSC maintenance medium. The iPSCs were cultured using a 12-well plate. The iPSC maintenance medium used was mTeSR Plus. The ROCK inhibitor was Y-27632. A concentration of the ROCK inhibitor was 10 μM. Confluence of the cultured iPSCs was 30% to 50%.

Step b. Induction and Differentiation of iPSCs into MSCs:

The iPSC maintenance medium for iPSCs in step a was replaced with a induction/differentiation medium for producing of mesenchymal stem cells. The cells were placed in a constant temperature incubator at 5% CO₂ and 37° C. for culture. The cells were designated as P0 at this time. When confluence reached 80% to 90%, supernatant was aspirated. Pre-warmed Ca²⁺ and Mg²⁺-free DPBS was added to wash the cells twice. Then, pre-warmed Tryple was added to digest the cells until the cells reached a single-cell state. Digestion was terminated. After centrifugation, the supernatant was removed. The cells were resuspended using 1 mL of the induction/differentiation medium for producing of mesenchymal stem cells. The cells were seeded at a density of 5×10⁴ cells/cm² into a well plate pre-coated with Laminin-521. The well plate was placed in a constant temperature incubator at 5% CO₂ and 37° C. for culture. The cells were designated as P1 at this time. According to the above steps, the cells were passaged and cultured to P3 passage. A composition of the induction/differentiation medium for producing of mesenchymal stem cells was: a serum-free basal medium for mesenchymal stem cells (a basal medium was selected from DMEM/F12 medium), containing 5% (v/v) human platelet lysate, 50 mM ascorbic acid, 1% (v/v) GlutaMax additive, 1% (v/v) NEAA, 1% (v/v) ITS-X supplement, 5 ng/mL IGF, 5 ng/mL bFGF, 5 ng/mL PDGF-BB, 5 μM TGF-β inhibitor (A8301), 5 μM GSK3β inhibitor (CHIR99021), and 8 μM ROCK inhibitor (Y-27632).

Step c. Expansion Passage Culture of iMSCs

When confluence of the P3 passage cells obtained in step b reached about 80% to 90%, the cells were digested again to the single-cell state. The cells were resuspended in 1 mL of an expansion medium for mesenchymal stem cells. The cells were seeded at a density of 0.9×10⁴ cells/cm² onto a plate pre-coated with the Laminin-521. The plate was placed in a 5% CO₂, 37° C. incubator for culture for 3-5 days. When the confluence of the cells reached about 80% to 90%, the cells were subjected to passage culture in a same manner as described above. The cells were passaged to P12 (Passage 12). P3 passage MSCs to P12 passage MSCs were obtained. A composition of the expansion medium for mesenchymal stem cells was: a serum-free basal medium for mesenchymal stem cells (a basal medium was selected from a DMEM/F12 medium), containing 5% (v/v) of human platelet lysate, 50 mM ascorbic acid, 1% (v/v) GlutaMax additive, 1% (v/v) NEAA, 1% (v/v) ITS-X supplement, 5 ng/mL of IGF, 5 ng/mL of bFGF, and 5 ng/mL of PDGF-BB.

### Effect Example 1

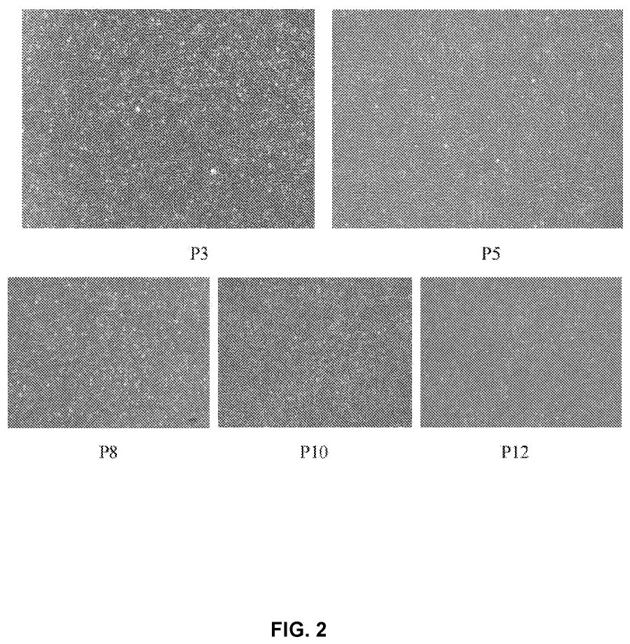

A morphology of the P3 passage and the subsequent passage MSCs prepared according to Example 2 was observed. Results are shown in FIG. 2. The P3 to P12 passage MSCs all exhibited a characteristic spindle-shaped morphology of MSCs arranged in parallel or spiral patterns. No signs of cellular senescence, such as an increased cell size or a reduced proliferation rate, were observed, and the cells were able to proliferate and be passaged stably.

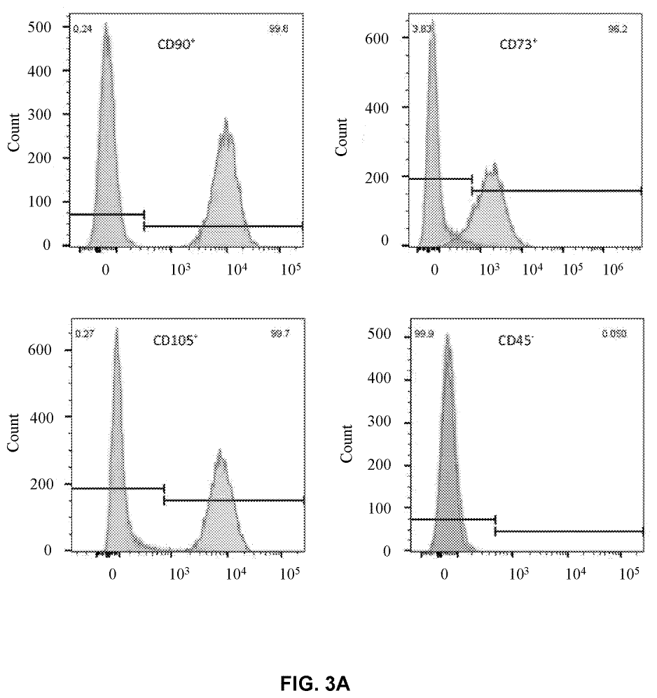

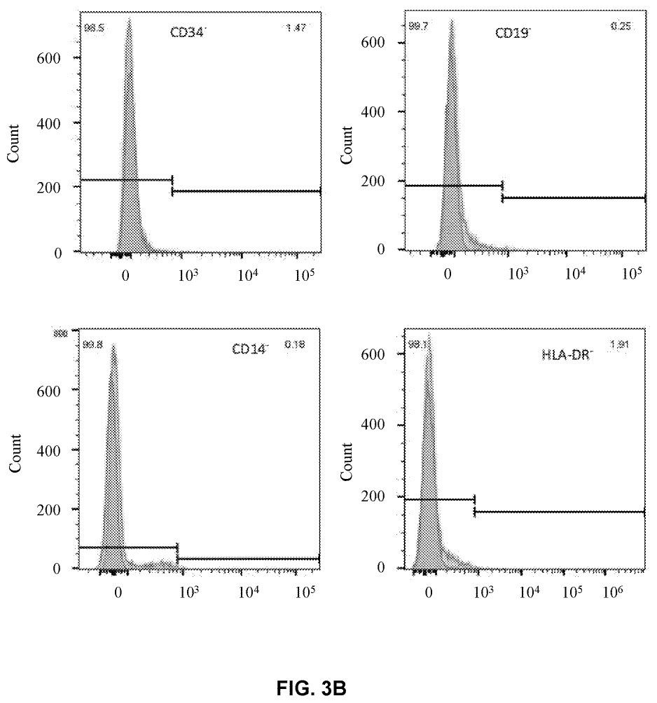

Meanwhile, P8 passage MSCs prepared in Example 2 were collected and analyzed by flow cytometry. Detection results are shown in FIG. 3. The MSCs exhibited expression levels greater than 95% for CD73, CD90, and CD105, indicating positive expression, while the expression levels of CD19, CD14, CD34, CD45, and HLA-DR were less than 2%, indicating negative expression. It should be specifically noted that, although the P8 passage cells were tested for flow cytometry analysis in this experimental experiment of the present disclosure, according to latest experimental data, the cells can be stably maintained for use up to passage P12.

### Effect Example 2

To verify an effect of iPSC gradually differentiating into iMSC in the induction/differentiation medium for producing of mesenchymal stem cells provided by the present disclosure, this experimental example further verified the above effect by detecting expression of related genes in related cells. Specific steps were as follows:

The experiment was divided into 3 groups: an iMSC group (P1, P3, P5, P7 passage MSCs prepared in Example 2 of the present disclosure), an iPSC group (prepared in Example 1 of the present disclosure), and an ADSC group (adipose MSCs). RNA was extracted from the above 3 groups of cells, respectively. Expression of OCT4, TWIST1, COL6A2, and SNAI2 was detected using a quantitative real-time polymerase chain reaction (qRT-PCR) manner. Specific steps were as follows:

Step a, RNA extraction: extraction was performed using an RNA purification kit (manufacturer: TransGen Biotech Co., Ltd., catalog number: ER101-01) to obtain a RNA sample.

Step b, reverse transcription: 1 g of the RNA sample was taken. Reverse transcription was performed according to requirements of a chain reaction reverse transcription kit (manufacturer: TransGen Biotech Co., Ltd., catalog number: AU341-02). A reverse transcription system is as follows:

| Component | Volume |
|---|---|
| RNA | 1 μg |
| 4 × RT mix | 4 μL |
| dd H2O | Make up to 15 μL |

The above samples were mixed and centrifuged. The samples were incubated at 42° C. for 15 min, and inactivated at 85° C. for 5 s. The obtained cDNA was diluted 2 times for subsequent reactions.

Step c, quantitative Real-Time PCR (qRT-PCR): The samples obtained above were subjected to qRT-PCR detection according to the following system. A reaction system is as follows:

| Component | Volume | |
|---|---|---|
| cDNA | 2 | μL |
| 2 × SYBR Mix | 10 | μL |
| Forward primer | 0.5 | μL |
| Reverse primer | 0.5 | μL |
| dd H₂O | 7 | μL |

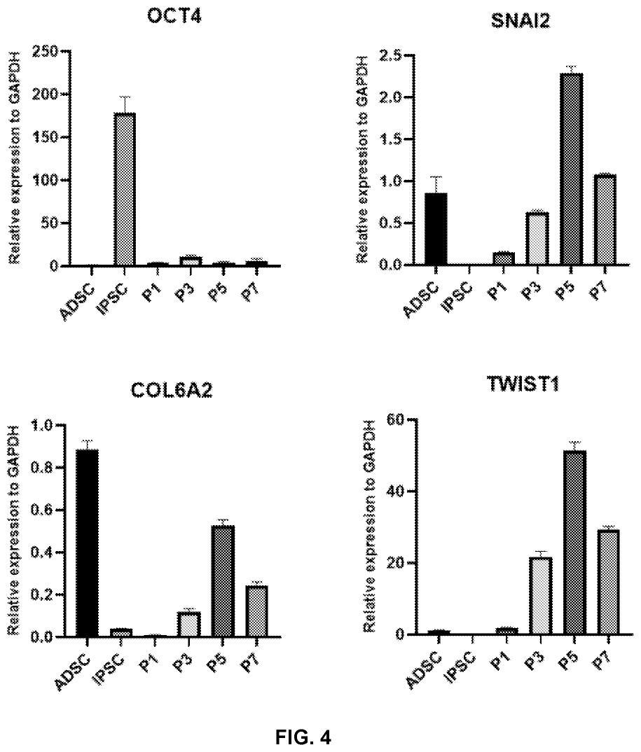

Detection results obtained through the above manner are shown in FIG. 4. According to the results shown in FIG. 4, MSCs of different passages exhibited significant expression of TWIST1, COL6A2, and SNAI2. According to gene expression results of P1, P3, P5, and P7 passage cells shown in FIG. 4, the cells had clearly undergone epithelial-to-mesenchymal transition (EMT). Combined with flow cytometry detection results of P8 passage cells shown in FIG. 3, it further indicates that the cells had differentiated into the iMSCs. OCT4 is a marker gene of the iPSC. When iPSCs successfully differentiated into iMSCs, cells of each passage exhibited a situation of not expressing the OCT4. As shown in FIG. 4, P1, P3, P5, and P7 passage cells almost did not express the OCT4 or expressed extremely trace amounts. This further indicates that, through the method for direct induction and differentiation provided by the present disclosure, the iPSCs can be successfully induced to differentiate into the iMSCs.

### Effect Example 3

Osteogenic and adipogenic differentiation capabilities of P8 passage MSCs prepared in Example 2 were further detected as follows:

Step a. Osteogenic Differentiation Identification:

P8 passage MSCs were seeded into a cell culture container at a suitable seeding density.

An appropriate amount of a pre-warmed fresh iMSC expansion medium was added. The container was shaken horizontally in a cross pattern three times. The container was placed in a 37° C., 5% CO₂ concentration, saturated humidity incubator. The container was shaken horizontally in the cross pattern three times again before culturing. The iMSCs spread and grew uniformly. After the culturing, when confluence of the iMSCs reached 80%-90%, the medium in the container was aspirated and replaced with an osteogenic differentiation induction medium, which was recorded as day 0. The medium was completely replaced every three days. The culture was continued until day 21. A composition of the osteogenic differentiation induction medium used was: an Alpha-MEM, containing 10% FBS, 1% Penicillin-Streptomycin, 10 mM β-glycerophosphate, 10 nM Dexamethasone, and 50 g/mL Ascorbic acid (L-ascorbic acid).

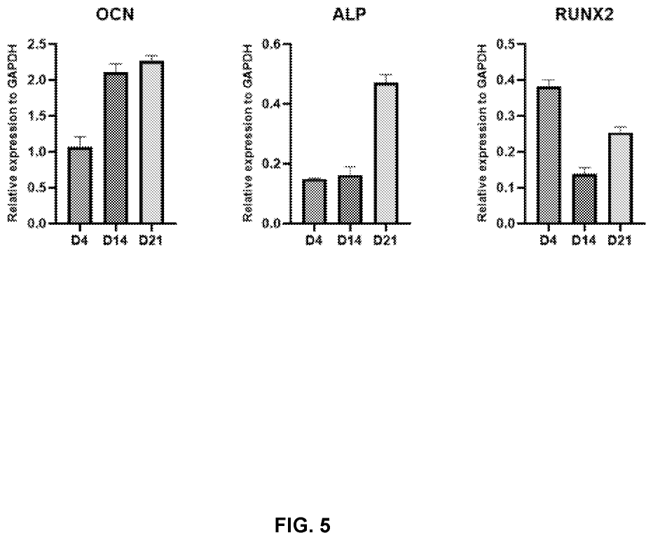

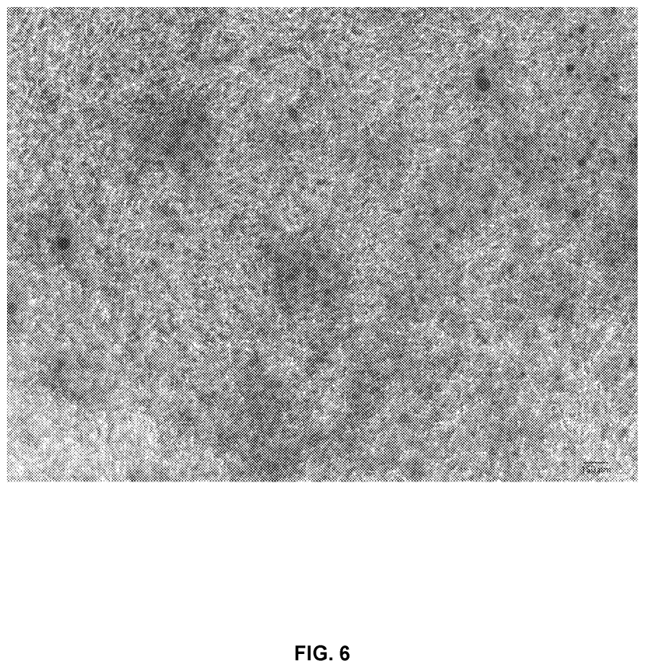

Differentiated cells cultured to day 4, day 14, and day 21 were observed and photographed under a light microscope. RNA of the differentiated cells was collected. Expression of osteogenic-specific genes RUNX2, ALP, and OCN was tested. Results are shown in FIG. 5. According to the results shown in FIG. 5, as a differentiation culture time increased, expression levels of specific genes ALP and OCN gradually increased. An expression level of a specific gene RUNX2 first decreased and then increased. This indicates that the differentiated iMSCs had ability to differentiate into osteocytes. After day 21, the osteogenic-differentiated iMSCs were washed with pure water. An appropriate volume of Alizarin Red working solution was added. The iMSCs were incubated at a room temperature in dark for 20-30 min. Excess staining solution was then aspirated and discarded. An appropriate volume of saline or DPBS was added to each well to keep the cells hydrated. The iMSCs were observed under a microscope and photographed. Results are shown in FIG. 6. According to the results shown in FIG. 6, obvious calcium nodule formation was observed.

Step b. Adipogenic Differentiation Identification:

P8 passage MSCs were seeded into a cell culture container at a suitable seeding density.

An appropriate amount of the pre-warmed fresh iMSC expansion medium was added. The container was shaken horizontally in the cross pattern three times. The container was placed in a 37° C., 5% CO₂ concentration, saturated humidity incubator. The container was shaken horizontally in the cross pattern three times again before culturing. The iMSCs spread and grew uniformly. After the culturing, when confluence of the iMSCs reached 80%-90%, the medium in the container was aspirated and replaced with an adipogenic differentiation induction medium, which was recorded as day 0. The medium was completely replaced every three days. The culturing was continued until day 21. A composition of the adipogenic differentiation induction medium used was: an Alpha-MEM, containing 10% FBS, 1% Penicillin-Streptomycin, 1 μM Dexamethasone, 0.5 mM 3-isobutyl-1-methylxanthine (IBMX), 0.2 mM Indomethacin, and 10 g/mL insulin.

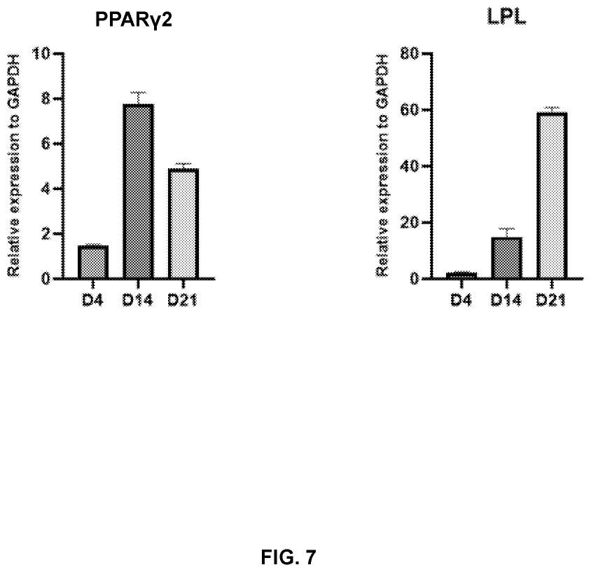

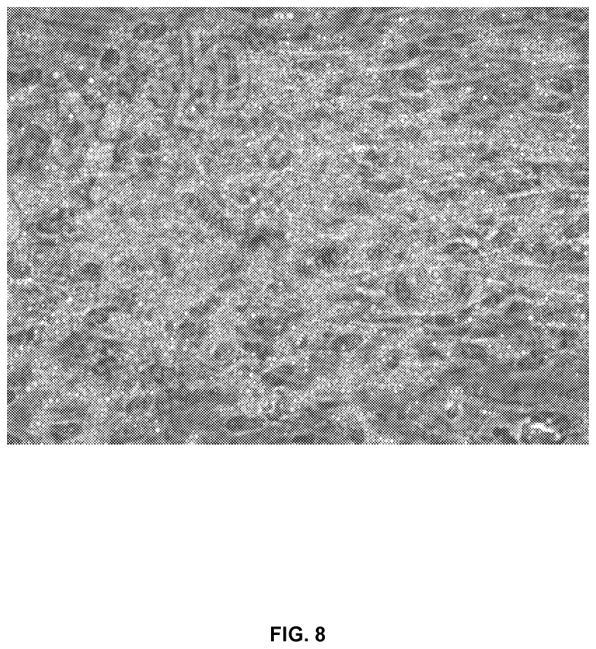

Differentiated cells cultured to day 4, day 14, and day 21 were observed and photographed under the light microscope. RNA of the differentiated cells was collected. Expression of adipogenic-specific genes PPARγ2 and LPL was tested. Results are shown in FIG. 7. According to the results shown in FIG. 7, as a differentiation culture time increased, an expression level of PPARγ2 first increased and then decreased. An expression level of LPL showed a trend of gradual increase. This indicates that the differentiated iMSCs had ability to differentiate into adipocytes. Simultaneously, during a normal differentiation process, it could be observed that the cells gradually became wider and shorter. Under high magnification, numerous round lipid droplets could be seen inside the cells. After day 21, the adipogenic-differentiated iMSCs were washed with the saline or the DPBS. The iMSCs were then washed once with 60% isopropanol solution to prevent residual saline or DPBS from causing precipitation of the staining solution. An appropriate volume of Oil Red 0 working solution was added to a differentiation group and a control group. The iMSCs were incubated at a room temperature in dark for 20-60 min. Excess staining solution was then aspirated and discarded. The iMSCs were washed with the saline or the DPBS until no background staining was observed. An appropriate volume of the saline or the DPBS was added to each well to keep the cells hydrated. The iMSCs were observed under a microscope and photographed. Results are shown in FIG. 8. According to the results shown in FIG. 8, obvious lipid droplet formation was observed.

### Comparative Example

This comparative example further studied effects of different induction and differentiation media used for culturing iPSCs on related performance of finally obtained MSCs. Details are as follows:

The following groups were set respectively:
Medium No. 1: an iMSC expansion medium.
Medium No. 2: 5 μM of a GSK3β inhibitor (CHIR99021) was added to an iMSC expansion medium;
Medium No. 3: 5 μM of a TGF-β inhibitor (A8301) was added to an iMSC expansion medium;
Medium No. 4: 8 μM of a ROCK inhibitor (Y-27632) was added to an iMSC expansion medium;
Medium No. 5: 5 μM of a GSK3β inhibitor (CHIR99021) and 5 μM of a TGF-β inhibitor (A8301) were added to an iMSC expansion medium;
Medium No. 6: 5 μM of a GSK3β inhibitor (CHIR99021), 5 μM of a TGF-β inhibitor (A8301), and 8 μM of a ROCK inhibitor (Y-27632) were added to an iMSC expansion medium;

The iMSC expansion medium has a composition including: a serum-free basal medium for mesenchymal stem cells (the basal medium is selected from a DMEM/F12 medium), 5% (v/v) human platelet lysate, 50 mM ascorbic acid, 1% (v/v) GlutaMax additive, 1% (v/v) NEAA, 1% (v/v) ITS-X supplement, 5 ng/mL of IGF, 5 ng/mL of bFGF, and 5 ng/mL of PDGF-BB;

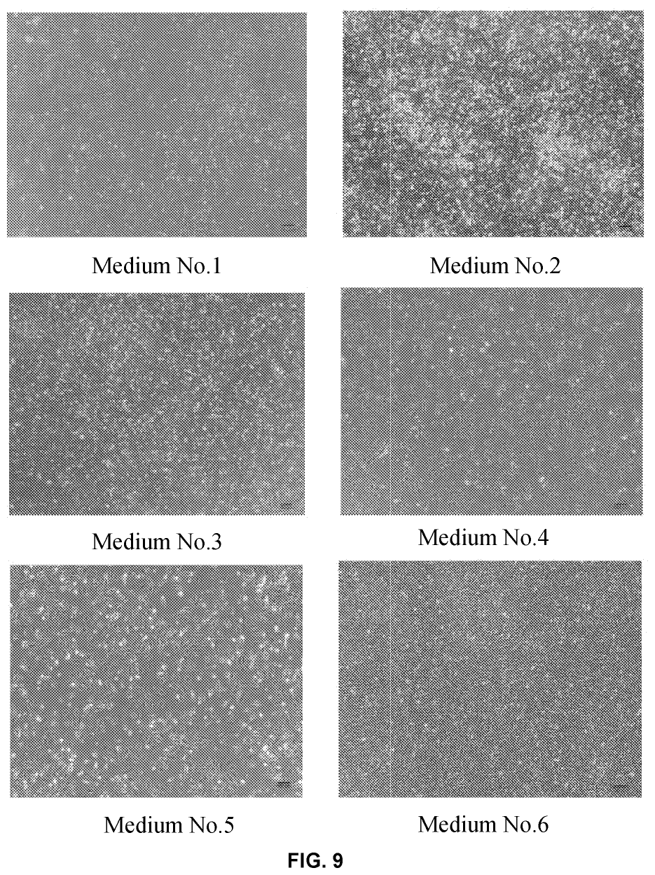

After passaging the iPSC prepared in Example 1, the old medium was removed. The iPSCs were divided into six groups. The six groups were respectively replaced with the six media described above. The cells were cultured in an incubator at 5% CO₂ and 37° C. A corresponding fresh medium for induction and differentiation was replaced daily. A cell morphology and proliferation were observed, and the cells were passaged when confluence of the cells reached 85% to 90%. During a differentiation process from the iPSCs to the iMSCs, the cell morphology was observed. Results are shown in FIG. 9. According to the results shown in FIG. 9, cells cultured in the Medium No. 1 exhibited almost no change in morphology and showed little proliferation. Cells cultured in the medium No. 2 gradually died during differentiation. Cells cultured in the medium No. 3 also showed almost no morphological changes. Cells cultured in the media No. 4 and the No. 5 showed no obvious proliferation when passaged to P1. Cells cultured in the medium No. 6 could be passaged to P2, and normal adherence was observed, with most cells exhibiting a short spindle-shaped morphology.

A further comparison was conducted to evaluate the effects of the following three groups of media for induction and differentiation on a differentiation of the iPSCs and a preparation of the MSCs. Details are as follows:
Group A medium: the Medium No. 6;
Group B medium: a basal medium E6 (brand: Gibco, catalog number: A1516401), added with 10 μM of a TGF-β inhibitor (SB431542), 2 μM of a GSK3β inhibitor (CHIR99021), and 10 ng/mL of bFGF;
Group C medium: a basal medium E6 (brand: Gibco, catalog number: A1516401), added with 0.1 mM of 2-mercaptoethanol, 1% of ITS-X supplement, 1.5 g/mL of L-AA-pi, 50 ng/mL of Epidermal Growth Factor (EGF), 2 μM of a GSK3β inhibitor (CHIR99021), 0.5 μM of a TGF-β inhibitor (A83-01), 1 μM of a TGF-β inhibitor (SB431542), 0.8 mM of Valproic Acid (VPA), 5 μM of a ROCK inhibitor (Y27632), and 10 ng/mL of Bone Morphogenetic Protein 4 (BMP4).

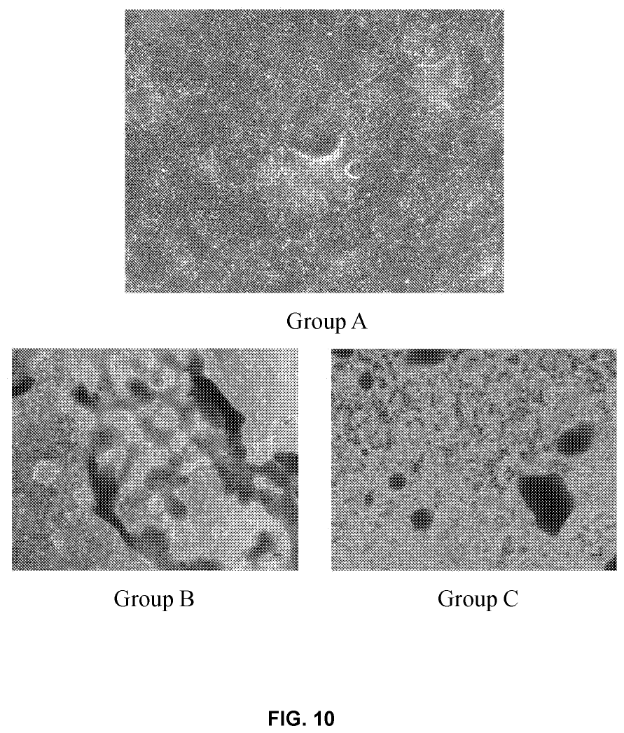

Single-cell iPSCs prepared in Example 1 were seeded and cultured for 2 days. When confluence reached about 30%, the iPSCs were divided into three groups. The three groups were respectively replaced with iPSC-iMSC induction and differentiation media of Group A, Group B, and Group C. A corresponding fresh medium was replaced daily. On day 6 of culture, a corresponding cell morphology was observed. Results are shown in FIG. 10. According to the results shown in FIG. 10, cells cultured in medium No. 6 of the group A exhibited a good differentiation state. Mesenchymal-like cells were observed under a microscope. In contrast, varying degrees of cell death occurred in the Group B and the Group C by day 6 of culture.

Combined with the results of the cell morphology and proliferation shown in FIG. 9 and FIG. 10, it further indicated that direct differentiation culture of the iPSCs using the induction/differentiation medium for producing of mesenchymal stem cells provided in the present disclosure can yield MSCs with excellent overall performance.

It is to be understood that the present disclosure is described byway of some embodiments. As is known to those skilled in the art, various changes or equivalent replacements may be made to these features and embodiments without departing from the spirit and scope of the present disclosure. In addition, under the teaching of the present disclosure, these features and embodiments may be modified to adapt to specific situations and materials without departing from the spirit and scope of the present disclosure. Therefore, the present disclosure is not limited by the specific embodiments disclosed herein. All embodiments falling within the scope of the claims of the present disclosure belong to the protection scope of the present disclosure.

What is claimed is:

1. A induction/differentiation medium for producing of mesenchymal stem cells, wherein the induction/differentiation medium is used for directly inducing differentiation of human induced pluripotent stem cells or embryonic stem cells into mesenchymal stem cells, and the induction/differentiation medium comprises: a mesenchymal stem cell basal medium and additives, wherein the additives include: 2 to 10 μM of transforming growth factor-β (TGF-β) inhibitor, 2 to 10 μM of glycogen synthase kinase-3β (GSK3β) inhibitor, and 5 to 10 μM of rho-associated coiled-coil containing protein kinase (ROCK) inhibitor.

2. The induction/differentiation medium according to claim 1, wherein the additives further include: human platelet lysate, ascorbic acid, GlutaMax additive, non-essential amino acids (NEAA), insulin-transferrin-selenium-ethanolamine (ITS-X) supplement, insulin-like growth factor (IGF), basic fibroblast growth factor (bFGF), and platelet-derived growth factor-BB (PDGF-BB).

3. The induction/differentiation medium according to claim 2, wherein: the human platelet lysate is at a volume concentration of 1 to 10%; the ascorbic acid is at a concentration of 30 to 70 mM; the GlutaMax additive is at a volume concentration of 0.5% to 2%; the NEAA is at a volume concentration of 0.5% to 2%; the ITS-X supplement is at a volume concentration of 0.5% to 2%; the IGF is at a concentration of 1 to 10 ng/mL; the bFGF is at a concentration of 1 to 10 ng/mL; and the PDGF-BB is at a concentration of 1 to 10 ng/mL.

4. The induction/differentiation medium according to claim 1, wherein: the TGF-β inhibitor includes at least one of SB431542, A-8301, or LY2157299; the GSK3β inhibitor includes at least one of CHIR99021, SB415286, or LY2090314; and the ROCK inhibitor includes at least one of Y-27632 or Blebbistatin.

5. The induction/differentiation medium according to claim 1, wherein the mesenchymal stem cell basal medium is a serum-free basal medium for mesenchymal stem cells; and the serum-free basal medium for mesenchymal stem cells includes at least one of high-glucose dulbecco's modified eagle medium (DMEM), alpha minimum essential medium (Alpha-MEM), or dulbecco's modified eagle medium/ham's f-12 nutrient mixture (DMEM/F12) medium.

6. A method for directly inducing and differentiating human induced pluripotent stem cells or embryonic stem cells into mesenchymal stem cells, the method comprising: subjecting the human induced pluripotent stem cells or the embryonic stem cells to passaging culture in the induction/differentiation medium according to claim 1, thereby directly inducing and differentiating the human induced pluripotent stem cells or the embryonic stem cells into the mesenchymal stem cells.

7. The method according to claim 6, comprising:
step a. culturing a cell culture of the human induced pluripotent stem cells or the embryonic stem cells; and
step b. replacing a medium in the cell culture with the induction/differentiation medium to perform passaging culture, thereby obtaining P3~P5 passage mesenchymal stem cells or a cell culture including the P3~P5 passage mesenchymal stem cells.

8. The method according to claim 7, wherein, in step a, the culturing the cell culture includes: culturing the human induced pluripotent stem cells or the embryonic stem cells in a xeno-free medium in a feeder-free culture manner.

9. The method according to claim 8, wherein the step of culturing the cell culture further includes: first culturing the human induced pluripotent stem cells or the embryonic stem cells normally in an induced pluripotent stem cell (iPSC) maintenance medium; when confluence of the cell culture reaches 80% to 90%, digesting the human induced pluripotent stem cells with Tryple into a single-cell suspension; resuspending the single-cell suspension in an iPSC maintenance medium; adding the ROCK inhibitor to the iPSC maintenance medium; after maintaining the ROCK inhibitor for 24 h, and replacing with a complete iPSC maintenance medium to obtain the cell culture at 30% to 50% confluence.

10. The method according to claim 9, wherein:
the iPSC maintenance medium includes at least one of Chemically Defined Essential 8 Medium (E8), StemFit Basic04, or mTeSR Plus;
the ROCK inhibitor includes at least one of Y-27632 or Blebbistatin; a matrix gel used in the culturing the cell culture includes at least one of Laminin-521, Vitronectin, or Matrigel; and
culturing conditions for the step of culturing the cell culture include: culturing in an incubator at 35-40° C., 4-6% CO₂, and 92-98% humidity.

11. The method according to claim 7, wherein, in step b, replacing the medium in the cell culture with the induction/differentiation medium to perform passaging culture includes: replacing the medium in the cell culture with the induction/differentiation medium; when confluence reaches 80%-90%, designating the cell culture as P0 passage mesenchymal stem cells; performing a digestion treatment on the P0 passage mesenchymal stem cells; resuspending the P0 passage mesenchymal stem cells after the digestion treatment using the induction/differentiation medium; continuing culturing to obtain P1 passage mesenchymal stem cells; and repeating the above passage culturing steps to obtain the P3~P5 passage mesenchymal stem cells;
wherein the digestion treatment includes: aspirating supernatant; adding Ca²⁺ and Mg²⁺-free dulbecco's phosphate buffered saline (DPBS) to wash the P0 passage mesenchymal stem cells; performing the digestion treatment on the P0 passage mesenchymal stem cells after washing; centrifuging to remove supernatant; and obtaining the P0 passage mesenchymal stem cells after the digestion treatment.

12. The method according to claim 7, further comprising: placing the P3~P5 passage mesenchymal stem cells or the cell culture including the P3~P5 passage mesenchymal stem cells in an expansion medium for mesenchymal stem cells to perform expansion passage culture, thereby obtaining the mesenchymal stem cells.

13. The method according to claim 12, wherein the expansion medium for mesenchymal stem cells includes: a mesenchymal stem cell basal medium and additives; wherein the additives include: human platelet lysate, ascorbic acid, GlutaMax additive, NEAA, ITS-X supplement, IGF, bFGF, and PDGF-BB;
wherein the mesenchymal stem cell basal medium is a serum-free basal medium for mesenchymal stem cells; and the serum-free basal medium for mesenchymal stem cells includes at least one of high-glucose DMEM, Alpha-MEM, or DMEM/F12 medium.

14. The method according to claim 13, wherein: the human platelet lysate is at a volume concentration of 1 to 10%; the ascorbic acid is at a concentration of 30 to 70 mM; the GlutaMax additive is at a volume concentration of 0.5% to 2%; the NEAA is at a volume concentration of 0.5% to 2%; the ITS-X supplement is at a volume concentration of 0.5% to 2%; the IGF is at a concentration of 1 to 10 ng/mL; the bFGF is at a concentration of 1 to 10 ng/mL; and the PDGF-BB is at a concentration of 1 to 10 ng/mL.

15. Mesenchymal stem cells prepared by the method according to claim 6,
wherein the mesenchymal stem cells are multipotent cells capable of differentiating into an adipocyte, an osteocyte, a chondrocyte, a myocyte, a neuron, and a cardiomyocyte;
wherein the mesenchymal stem cells are capable of expressing cell surface markers Cluster of Differentiation (CD) 29, CD73, CD90, CD105, CD166, and CD44; and
wherein the mesenchymal stem cells do not express cell surface markers Human Leukocyte Antigen-DR isotype (HLA-DR), CD34, CD45, CD19, and CD14.

16. The mesenchymal stem cells prepared by the method according to claim 6, wherein the mesenchymal stem cells are P5 or higher passage mesenchymal stem cells; in the mesenchymal stem cells, a proportion of cells expressing CD29, CD44, CD73, CD90, CD105, and CD166 is not less than 95%; and in the mesenchymal stem cells, a proportion of cells expressing HLA-DR, CD34, CD45, CD19, and CD14 is not more than 2%.
